(19) United States
(12) Patent Application Publication
(10) Pub. No.: US 2023/0393123 A1
(43) Pub. Date: Dec. 7, 2023

(54) CONCENTRATION ESTIMATION KIT AND CONCENTRATION ESTIMATION METHOD

(71) Applicant: Tianma Japan, Ltd., Kawasaki (JP)

(72) Inventors: Ayuko IMAI, Kawasaki (JP); Ken SUMIYOSHI, Kawasaki (JP)

(73) Assignee: Tianma Japan, Ltd., Kawasaki (JP)

(21) Appl. No.: 18/327,118

(22) Filed: Jun. 1, 2023

(30) Foreign Application Priority Data
Jun. 1, 2022 (JP) 2022-089389

(51) Int. Cl. G01N 33/53; C07K 16/44; G01N 33/532

(52) U.S. Cl. CPC G01N 33/5308; G01N 33/532; C07K 16/44

(57) ABSTRACT

A concentration estimation kit includes a derivatization reagent that derivatizes at least a portion of an antigen included in a mixed specimen, and that is for obtaining a plurality of samples having different derivatization rates, the derivatization rate being a ratio of a derivatized antigen relative to the antigen included in the specimen; an antibody that binds to the antigen; and the antigen modified with a dye.

### REFERENCE TO RELATED APPLICATIONS

This application claims the benefit of Japanese Patent Application No. 2022-89389, filed on Jun. 1, 2022, the entire disclosure of which is incorporated by reference herein.

### FIELD OF THE INVENTION

The present disclosure generally relates to a concentration estimation kit and a concentration estimation method.

### BACKGROUND OF THE INVENTION

The fluorescence polarization immunoassay (FPIA) method is an immunoassay method that uses fluorescence. The degree of fluorescence polarization measured in FPIA is proportional to the effective volume of the substance to be measured. Japanese Unexamined Patent Application Publication No. H03-103765 describes an FPIA that uses the feature of the degree of fluorescence polarization changing due to a specific antigen-antibody reaction between a reagent, in which an antibody is immobilized on a substance with a greater molecular weight than the antibody, and a fluorescently labeled antigen.

The FPIA uses an antibody that specifically binds to the antigen that is the substance to be measured. To acquire such an antibody, the substance to be measured must have immunogenicity, which is the activity that induces antibody production. When a hapten, which binds to the antibody but is not immunogenic on its own due to its low molecular weight, is the substance to be measured, the hapten must be rendered immunogenic to acquire the antibody. For example, Japanese Unexamined Patent Application Publication No. S51-104029 discloses that, in order to quantify antipyrine, an assay based on an antigen-antibody reaction using an antibody prepared using an antipyrine derivative, obtained by binding antipyrine to serum albumin, as an antigen.

In the FPIA, changes in the degree of fluorescence polarization corresponding to the binding of the antigen and the antibody are detected, but the range of the concentration of the antigen that binds to the antibody, that is, the substance to be measured, is limited. Consequently, in the FPIA, the range of concentration of the substance to be measured estimable on the basis of a calibration curve based on measured values of the degree of fluorescence polarization depends on the affinity between the substance to be measured and the antibody. When there is not a plurality of antibodies for which affinity with the substance to be measured differs, the estimable range of the concentration of the substance to be measured is limited.

### SUMMARY OF THE INVENTION

A concentration estimation kit according to a first aspect of the present disclosure includes:

a derivatization reagent that derivatizes at least a portion of an antigen included in a mixed specimen, and that is for obtaining a plurality of samples having different derivatization rates, the derivatization rate being a ratio of a derivatized antigen relative to the antigen included in the specimen;

an antibody that binds to the antigen; and

the antigen modified with a dye.

A concentration estimation method according to a second aspect of the present disclosure includes:

mixing a specimen including an antigen and a derivatization reagent that derivatizes at least a portion of the antigen included in the specimen, and obtaining a plurality of samples having different derivatization rates, the derivatization rate being a ratio of a derivatized antigen relative to the antigen included in the specimen;

mixing each of the samples, an antibody that binds to the antigen, and the antigen modified with a dye to obtain a plurality of solutions to be measured; and

measuring a degree of polarization of each of the solutions to be measured. It is to be understood that both the foregoing general description and the following detailed description are exemplary and explanatory and are not restrictive of this disclosure.

### BRIEF DESCRIPTION OF THE DRAWINGS

A more complete understanding of this application can be obtained when the following detailed description is considered in conjunction with the following drawings, in which:

FIG. 1 is a drawing illustrating a ratio of an antigen bound to an antibody relative to the antigen when changing a concentration of the antibody and a derivatization rate;

FIG. 2 is a drawing illustrating a degree of polarization calculated from the derivatization rate;

FIG. 3 is a drawing schematically illustrating a multi-well plate including a tracer that is the antigen modified with a dye, and a derivatization reagent having a substance amount that differs by row;

FIG. 4 is a drawing illustrating the configuration of a degree of fluorescence polarization measuring device according to the examples;

FIG. 5 is a drawing illustrating microchannels in an effective field of view; and

FIG. 6 is a drawing illustrating a degree of polarization relative to a histamine concentration of samples having different added amounts of an acylating reagent.

### DETAILED DESCRIPTION

Embodiments of the present disclosure are described while referencing the drawings. Note that the present disclosure is not limited by the embodiments and drawings described below. Additionally, note that, in the following embodiments, the expressions “having” and “including”, or “containing” also include the meanings of “comprising” or “constituted from.”

A concentration estimation kit according to the present embodiment is a kit for an FPIA in which the binding of an antibody to a specific antigen is used to estimate the concentration of the antigen, which is a substance to be measured in a specimen. To facilitate description, in the following, the antigen in the specimen is referred to as the “substance to be measured.” The specimen is not particularly limited provided that the specimen is a substance to be inspected or analyzed. Examples of the specimen include cells, tissues, cell culture supernatants, cell extracts, tissue extracts, body fluids such as blood, saliva, urine and lymph obtained from human or non-human animals, biological samples such as nasal or nasopharyngeal swabs, beverages, foods, cleaning liquids for objects, and the like.

The concentration estimation kit according to the present embodiment includes a derivatization reagent, an antibody that binds to the substance to be measured, and an antigen modified with a dye. The derivatization reagent derivatizes at least a portion of the substance to be measured included in the mixed specimen. Here, the term “derivatization” means using, in addition to hydrogen atoms constituting the substance to be measured, a functional group such as a hydroxyl group, an amino group, a carboxyl group, a mercapto group, a carbonyl group, and a thiol group to add a substituent to the substance to be measured. Examples of the derivatization include silylation, acylation, esterification, oximation, and the like. The derivatization may take the form of using a known cross-linking agent to add a substituent to the substance to be measured. The substituent is any substituent that can be substituted by a known method for the atoms constituting the substance to be measured. Examples of the substituent include an acyl group, an alkyl group, and the like, of which the acyl group is preferable. When the acyl group is to be added to the substance to be measured, examples of the derivatization reagent include an acylating reagent (acylating agent) that replaces hydrogen atoms such as hydroxyl groups, amino groups, and mercapto groups of the substance to be measured with acyl groups (RCO-). Examples of the acylating reagent include acid chloride, acid anhydride, ketene, carboxylic acid, and the like, and specific examples of the acylating reagent include trifluoroacetic anhydride, imidazole trifluoroacetate, 4-chlorobutyryl chloride, and the like. Examples of a silylation reagent include hexamethyldisilazane (HMDS), N-trimethylsilylimidazole, and the like. Examples of an esterification reagent include acid-alcohol, N,N-dimethylformamide, dimethylacetal, an on-column methylating agent, diazomethane, and the like. Examples of an oximation reagent include pentafluorobenzyl, hydroxyamine hydrochloride, and the like. Examples of the cross-linking agent include aldehydes, ketones, and the like. In one example, the cross-linking agent is glutaraldehyde.

The derivatization reagent is used to obtain a plurality of samples having different derivatization rates. Here, the derivatization rate is a ratio of the derivatized substance to be measured relative to the substance to be measured included in the specimen. The derivatization rate is dependent on the amount (substance amount) of the derivatization reagent relative to the amount (substance amount) of the substance to be measured included in the specimen. Provided that the derivatization reagent is in the form of a plurality of solutions having different substance amounts of the derivatization reagent, when estimating the concentration of the substance to be measured in identical specimens, a plurality of samples having different derivatization rates can be obtained by mixing each of the specimens having the same volume and a plurality of solutions having different substance amounts of the derivatization reagent. For example, the plurality of solutions having different substance amounts of the derivatization reagent may include a solution for which the substance amount is a minimum value M, and a plurality of solutions for which the substance amount of the derivatization reagent is a positive real multiple of M and the substance amounts differ. The derivatization reagent may be a plurality of solutions that includes the derivatization reagent at identical concentrations and has different volumes, or may be a plurality of solutions that includes the derivatization reagent at different concentrations and has identical volumes.

The concentration estimation kit may include a non-derivatization reagent that is used to obtain a sample having a derivatization rate of 0. When the concentration estimation kit includes the plurality of solutions that contains the derivatization reagent at identical concentrations and different volumes, the concentration estimation kit may include a non-derivatization reagent that contains the same solvent as the solutions containing the derivatization reagent and that does not contain the derivatization reagent. When the concentration estimation kit includes the plurality of solutions that contains the derivatization reagent at different concentrations and identical volumes, the concentration estimation kit may include a non-derivatization reagent that contains the same solvent as the solutions containing the derivatization reagent, at an identical volume, and does not contain the derivatization reagent.

The antibody is not limited provided that the antibody specifically binds to the substance to be measured. Examples of the antibody include monoclonal antibodies, multispecific antibodies, bifunctional antibodies, human antibodies, humanized antibodies, antibodies from birds such as chickens, non-primates such as camels, non-human mammals, and other animals, recombinant antibodies, chimeric antibodies, single chain Fv, single chain antibodies, single domain antibodies, Fab fragments, F(ab′) fragments, F(ab′)2 fragments, disulfide-bonded Fv, anti-idiotypic antibodies, dual domain antibodies, dual variable domain antibodies, and the like.

The substance to be measured is not particularly limited provided that it is possible to change the affinity with the antibody by the derivatization with the derivatization reagent. Taking the efficiency of the derivatization into consideration, it is preferable that the substance to be measured is a low molecular weight substance rather than a high molecular weight substance such as a protein or the like. It is preferable that the substance to be measured is a hapten. A hapten is a substance that binds to antibodies, but does not exhibit immunogenicity, an activity that induces antibody production, on its own due to having a low molecular weight. Examples of the hapten include histamine, gamma-aminobutyric acid (GABA), dopamine, thyroid hormones, steroid hormones, and the like. More specifically, the thyroid hormones are triiodothyronine, thyroxine, 3,5-diiodo-L-thyronine, and the like. The steroid hormones are estrone, estradiol, estriol, progesterone, cortisol, testosterone, dehydroepiandrosterone sulfate, and the like. The hapten may be a low molecular weight peptide hormone, a catecholamine, a lemma enzyme vitamin, a drug, an antibiotic, a metabolite thereof, or the like.

The hapten becomes an immunogenic complete antigen by binding with an immunogenic substance such as a protein or the like. Examples of the immunogenic substance include immunogenic proteins, polypeptides, carbohydrates, polysaccharides, lipopolysaccharides, nucleic acids, and the like. The immunogenic substance is preferably a protein or a polypeptide, examples thereof including bovine serum albumin (BSA), keyhole limpet hemocyanin (KLH), and thyroglobulin.

When the substance to be measured is the hapten, it is preferable that the antibody is an antibody whose immunogen is a hapten derivative in which the immunogenic substance is bound to the hapten via a linker. The linker is a grouping introduced between the immunogenic substance and the hapten Examples of the linker include linkers including amides, disulfides, thioethers, hydrazones, hydrazides, imines, oximes, ureas, thioureas, amidines, amines, sulfonamides, and the like.

The antibody whose immunogen is a hapten derivative ((hapten)-(linker)-(immunogenic substance)) can be acquired by a known method. When acquiring the antibody, typically, it is sufficient that a host animal such as a rabbit, goat, mouse, guinea pig, horse, or the like is injected with the immunogen. To enhance the immunogenicity, it is preferable that a mixture of the immunogen and an adjuvant is injected. The immunogen may be further injected at regular or irregular intervals at the same site or different sites of the host animal. The antibody titer is appropriately evaluated, and the antibodies that specifically bind to the hapten derivative can be recovered by collecting blood from the host animal, or the like.

When the substance to be measured is the hapten and the antibody whose immunogen is a hapten derivative is used, it is preferable that the derivatization reagent imparts, to the hapten in the analyte, a structure identical to at least a portion of the linker. For example, when the linker contains an acyl group, it is sufficient that a derivatization reagent that imparts an acyl group to the hapten is used.

The antigen of the concentration estimation kit according to the present embodiment is modified with a dye, and functions as a tracer in the immunoassay. In the following, the antigen modified with the dye of the concentration estimation kit is also referred to as “tracer.” A fluorochrome that emits fluorescence is preferable as the dye. Each fluorochrome has a fluorescence lifetime. It is sufficient that a fluorochrome with a fluorescence lifetime of 1 to 10 ns, a fluorochrome with a fluorescence lifetime of greater than 10 ns to 200 ns, or a fluorochrome with a fluorescence lifetime of greater than 200 ns to 3000 ns is appropriately selected in accordance with the molecular weight and the like of the substance to be measured. Examples of the fluorochrome with a fluorescence lifetime of 1 to 10 ns include fluorescein compounds such as indolenine, chlorotriazinylaminofluorescein, 4′-aminomethylfluorescein, 5-aminomethylfluorescein, 6-aminomethylfluorescein, 6-carboxyfluorescein, 5-carboxyfluorescein, 5-aminofluorescein, 6-aminofluorescein, thioureafluorescein, methoxytriazinylaminofluorescein, and the like; rhodamine derivatives such as rhodamine B, rhodamine 6G, rhodamine 6GP, and the like; and, as registered trademarks or trade names, Alexa Fluor series such as Alexa Fluor 488, BODIPY series, DY series, ATTO series, Dy Light series, Oyster series, HiLyte Fluor series, Pacific Blue, Marina Blue, Acridine, Edans, Coumarin, DANSYL, FAN, Oregon Green, Rhodamine Green-X, NBD-X, TET, JOE, Yakima Yellow, VIC, HEX, R6G, Cy3, TAMRA, Rhodamine Red-X, Redmond Red, ROX, Cal Red, Texas Red, LC Red 640, Cy5, Cy5.5, and LC Red 705. Examples of the fluorochrome with a fluorescence lifetime of greater than 10 ns to 200 ns include naphthalene derivatives such as dialkylaminonaphthalenesulfonyl and the like, and pyrene derivatives such as N-(1-pyrenyl)maleimide, aminopyrene, pyrenebutanoic acid, alkynylpyrene, and the like. Examples of the fluorochrome with a fluorescence lifetime of greater than 200 ns to 3000 ns include metal complexes such as platinum, rhenium, ruthenium, osmium, europium, and the like.

To modify the antigen with the dye, it is sufficient that, for example, the dye and the antigen are directly covalently bonded or bonded via a linker such as oligoethylene glycol, an alkyl chain, or the like. When the antigen is the hapten, and the antibody whose immunogen is a hapten derivative is used, it is preferable that the dye is bonded to the hapten via a structure identical to at least a portion of the linker interposed between the hapten and the immunogenic substance in the hapten derivative. For example, when the linker interposed between the hapten and the immunogenic substance in the hapten derivative contains an acyl group, it is preferable that the linker interposed between the dye and the antigen contains an acyl group.

The dye has a functional group that can bind to the carboxyl group, the amino group, the hydroxyl group, the thiol group, the phenyl group, and the like of the antigen. The antigen can be labeled with the dye by reacting the respective functional groups of the dye and the antigen under known conditions. Note that the number of molecules of the dye that modifies one molecule of the antigen can be selected as desired. It is preferable that there is one molecule or more of the dye, and may be from two to five molecules, per one molecule of the antigen.

Next, an example is described of a concentration estimation method according to the present embodiment that uses the concentration estimation kit described above. The concentration estimation method includes a sample preparation step, a mixing step, and a measuring step. In the sample preparation step, the specimen containing the substance to be measured and the derivatization reagent described above are mixed, and a plurality of samples having different derivatization rates is obtained. The plurality of samples may be obtained by mixing the specimen and each of the plurality of solutions that contains the derivatization reagent at identical concentrations and different volumes, or may be obtained by mixing the specimen and each of the plurality of solutions that contains the derivatization reagent at different concentrations and identical volumes. For example, it is sufficient that identical amounts of the specimen are dispensed into each well of a multi-well plate, and each solution is added to each well.

In the mixing step, each sample, the antibody, and the tracer are mixed, and a plurality of solutions to be measured is obtained. Provided that the multi-well plate described above is used, it is sufficient that the antibody and the tracer are added to each well in which each sample is present.

In the measuring step, the degree of polarization of each of the solutions to be measured is measured. FPIA uses degree of polarization changes based on molecular weight changes of the tracer caused by the tracer bonding to the antibody to form a tracer-antibody conjugate. The dye in the solution emits polarized fluorescence on the same plane when maintaining a steady state in the excited state but, when rotated in Brownian motion while in the excited state, emits fluorescence on a plane different than the plane of excitation and, consequently, the fluorescence is depolarized. The degree of fluorescence polarization indicates how much the fluorescent molecules rotate in a period from excitation to when fluorescence is emitted. Molecules with low molecular weight rotate vigorously in solution due to Brownian motion and, as such, the degree of polarization is low. Molecules with high molecular weight have weak Brownian motion and, as such, the degree of polarization rises. For example, in a solution obtained by mixing an antibody B that specifically binds to a substance to be measured A, and a tracer C obtained by labeling the substance to be measured A with a fluorochrome, the substance to be measured A and the tracer C competitively react in the solution with the antibody B. As such, when the concentration of the substance to be measured A is high, the amount of the substance to be measured A bound to the antibody B increases (the amount of the tracer C bound to the antibody B decreases), and the amount of the free tracer C not bound to the antibody B increases. Meanwhile, when the concentration of the substance to be measured A is low, the substance to be measured A bound to the antibody B decreases (the tracer C bound to the antibody B increases), and the free tracer C not bound to the antibody B decreases. When there is a difference between the mass of the free tracer C and the mass of the conjugate formed by the tracer C binding to the antibody B, the concentration of the substance to be measured A can be measured using the change in the degree of polarization as an index.

In FPIA, the molecular weight change caused by the bonding of the tracer to the substance to be measured is measured as the temporal change in the molecular orientation. A desired polarization measuring device may be used to measure the degree of polarization. It is sufficient that the degree of polarization is measured a predetermined amount of time after the end of the reaction. To quantify the substance to be measured, it is sufficient that a calibration curve, obtained in advance by performing the same operation as above using a solution containing the substance to be measured at a known concentration, is created and compared against the measured values of the samples.

With the concentration estimation kit and the concentration estimation method according to the present embodiment, the degree of polarization of each of the solutions to be measured prepared from the plurality of samples having different derivatization rates is measured to estimate the concentration of the substance to be measured in the specimen. When Ag_A is the derivatized substance to be measured, Ag_B is the underivatized substance to be measured, Ab is the antibody, the conjugate of Ag_A and the antibody is Ag_A-Ab, and the conjugate of Ag_B and the antibody is Ag_B-Ab, the binding constant KA between Ag_A and Ab and the binding constant KB between Ag_B and Ab are expressed as follows.

$$Ag\_A + Ab ⇄ Ag\_A - Ab {K}_{A} = \frac{[ Ag\_A - Ab ]}{[ Ag\_A ] [ Ab ]} [ 1 / M ]$$

$$Ag\_B + Ab ⇄ Ag\_B - Ab {K}_{B} = \frac{[ Ag\_B - Ab ]}{[ Ag\_B ] [ Ab ]} [ 1 / M ]$$

When BF_A and BF_B are respectively a bound/free ratio of Ag_A and a bound/free ratio of Ag_B, the following is obtained.

$${K}_{A} = \frac{[ Ag\_A - Ab ]}{[ Ag\_A ] [ Ab ]} = \frac{BF\_A}{[ Ab ]}$$

$${K}_{B} = \frac{[ Ag\_B - Ab ]}{[ Ag\_B ] [ Ab ]} = \frac{BF\_B}{[ Ab ]}$$

$$\frac{{K}_{B}}{{K}_{A}} = \frac{BF\_B}{BF\_A} = {r}_{BA}$$

When pA, pB, and q, are respectively an introduction amount (M) of Ag_A, an introduction amount (M) of Ag_B, and an introduction amount (M) of Ab, BF_A can be calculated using the following cubic equation.

$$\frac{[ Ag\_A - Ab ]}{{p}_{A}} = \frac{[ Ag\_A - Ab ]}{⌈ Ag\_A ⌉ + [ Ag\_A - Ab ]} = \frac{[ Ag\_A - Ab ] / ⌈ Ag\_A ⌉}{1 + [ Ag\_A - Ab ] / ⌈ Ag\_A ⌉} = \frac{BF\_A}{1 + BF\_A} \qquad Equation 3$$

$$\frac{[ Ag\_B - Ab ]}{{p}_{B}} = \frac{[ Ag\_B - Ab ]}{⌈ Ag\_B ⌉ + [ Ag\_B - Ab ]} = \frac{[ Ag\_B - Ab ] / ⌈ Ag\_B ⌉}{1 + [ Ag\_B - Ab ] / ⌈ Ag\_B ⌉} = \frac{BF\_B}{1 + BF\_B}$$

$$\begin{matrix}[ Ab ] = q - [ Ag\_A - Ab ] - [ Ag\_B - Ab ] \\ = q - {p}_{A} \frac{BF\_A}{1 + BF\_A} - {p}_{B} \frac{BF\_B}{1 + BF\_B} = \\ q - {p}_{A} ( BF\_A ) / ( 1 + BF\_A ) - {p}_{B} {r}_{BA} ( BF\_A ) / ( 1 + {r}_{BA} * BF\_A ) \\ = BF\_A / {K}_{A}\end{matrix}$$

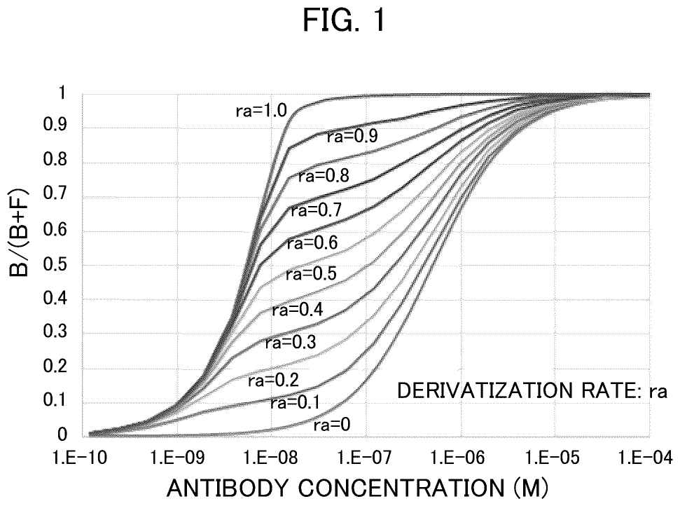

Estimating, on the basis of the cubic equation, the ratio of antigen bound to antibody relative to the antigen (total amount of the derivatized antigen and the non-derivatized antigen) while changing the derivatization rate and antibody concentration revealed that, as illustrated in FIG. 1, when the derivatization rate is changed, the affinity of the antigen to the antibody changes (K_{A}: 2E+9M⁻¹, K_{B}: 2E+6M ⁻¹ and total antigen amount (p_{A}+p_{B}): 1E-8M).

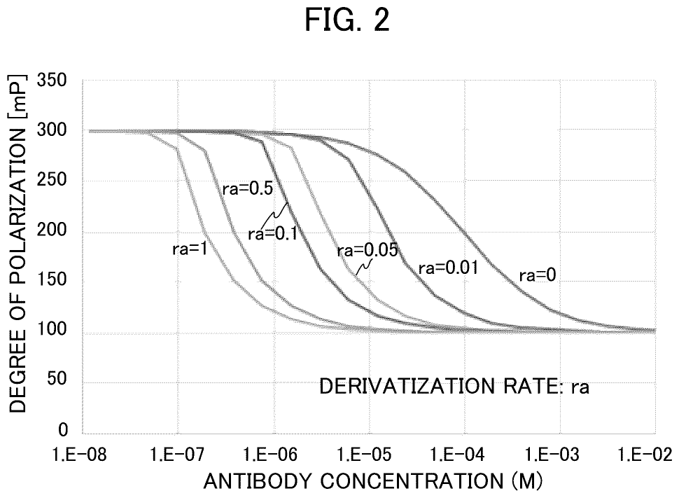

In FPIA, the degree of polarization of the tracer is measured. It is assumed that the binding constant of the tracer to the antibody is the same as the binding constant K_{A} of the derivatized antigen. The B/F ratio of the tracer is equivalent to the B/F ratio (BF_A) of the derivatized antigen. When Fh is the degree of polarization when the tracer is bound to the antibody and Fl is the degree of polarization when the tracer is free, a degree of polarization P of the tracer in the FPIA system is expressed by P=(Fh×BF_A+Fl)/(1+BF_A). FIG. 2 illustrates the degree of polarization calculated from the derivatization rate for a case in which K_{A}, K_{B}, the antibody concentration, the tracer concentration, Fh, and Fl are respectively 2E+9M⁻¹, 2E+6M⁻¹, 1E-7M, 1E-8M, 300 mP, and 100 mP. FIG. 2 demonstrates that, when the derivatization rates are different, the range of the antigen concentration in which the degree of polarization changes, changes.

According to the concentration estimation kit according to the present embodiment, the affinity of the substance to be measured to the antibody can be changed by measuring the degrees of polarization of a plurality of samples that contain the substance to be measured and that have different derivatization rates. In FPIA, the range of measurable concentrations depends on the affinity of substance to be measured to the antibody and, as such, the estimable range of the concentration of the substance to be measured can be expanded.

Note that a configuration is possible in which the concentration estimation kit includes a multi-well plate, wherein the derivatization reagent is immobilized in each well of the multi-well plate at different substance amounts. The derivatization reagent can be immobilized in the wells by a known method. For example, it is sufficient that the solution containing the derivatization reagent is added to the wells, and the solvent is removed by drying or the like.

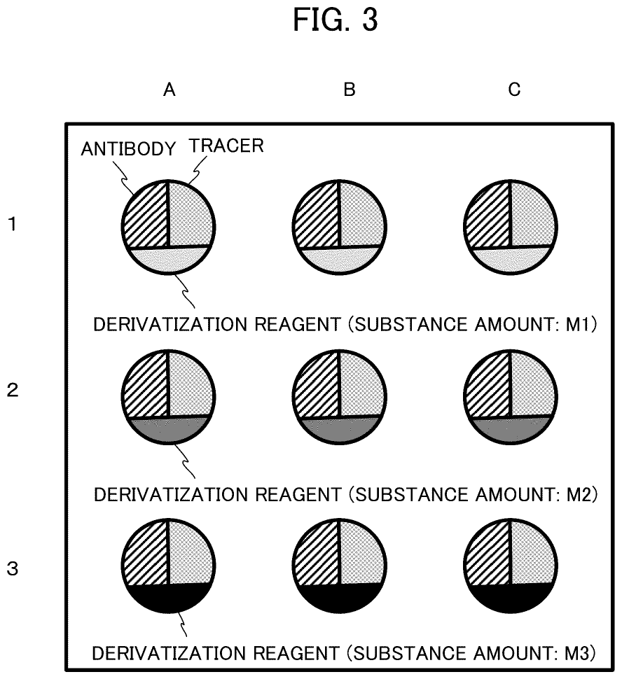

FIG. 3 schematically illustrates the multi-well plate described above. The multi-well plate includes wells arranged 3×3. The antibody, the tracer, and the derivatization reagent are immobilized in each well. The substance amounts of the immobilized antibody and the tracer are the same in all of the wells. However, the substance amount of the immobilized derivatization reagent differs by row, and the substance amounts of the derivatization reagent immobilized in wells of the first row, the second row, and the third row are respectively M1, M2, and M3.

Using column A as an example, by adding identical volumes of the specimen to each of the three wells of column A, it is possible to derivatize the substance to be measured contained in the specimen in accordance with the substance amount of the derivatization reagent. By using the multi-well plate described above, it is possible to easily obtain a plurality of samples having different derivatization rates. The multi-well plate includes column B and column C that have different substance amounts of the derivatization reagent similar to column A and, as such, is useful for tests of different specimens, duplicate tests, triplicate tests, and the like. Note that the number of wells of the plate may be greater than nine, and the numbers of the rows and the columns are appropriately set in accordance with the number of wells.

### EXAMPLES

Hereinafter, the present disclosure is described in further detail using examples, but the present disclosure is not limited to these examples.

Histamine (manufactured by Fuji Film Wako Pure Chemical Corp.) was modified with 5/6-TAMRA (Rhodamine) to obtain a histamine tracer. The histamine tracer was dissolved in pure water to obtain a 2nM solution. An anti-histamine antibody (manufactured by Progen Biotechnik) was diluted with a phosphate buffer (PBS) to prepare a 21 nM solution. The histamine (manufactured by Fuji Film Wako Pure Chemical Corp.) was dissolved in pure water to prepare a 33 mg/mL solution. 100 μL of the obtained histamine solution was introduced into each of four test tubes, and the acylating reagent included in the RIDA screen histamine ELISA kit (manufactured by R-Biopharm) was added to each test tube. Added amounts of the acylating reagent were 25 μL (sample 1), 10 μL (sample 2), and μL (sample 3). The acylating reagent was not added to one of the test tubes (sample 4).

An operation of diluting samples 1 to 4 ten-fold with water was carried out eight times. Thus, nine levels of samples were obtained for samples 1 to 4. 25 ∞L of each of the obtained nine levels of samples, 25 μL of the histamine tracer, and 25 μL of the antibody solution were mixed and allowed to rest in a light-shielded environment at room temperature for 60 minutes and, then, the degree of fluorescence polarization of each of the mixed solutions was measured.

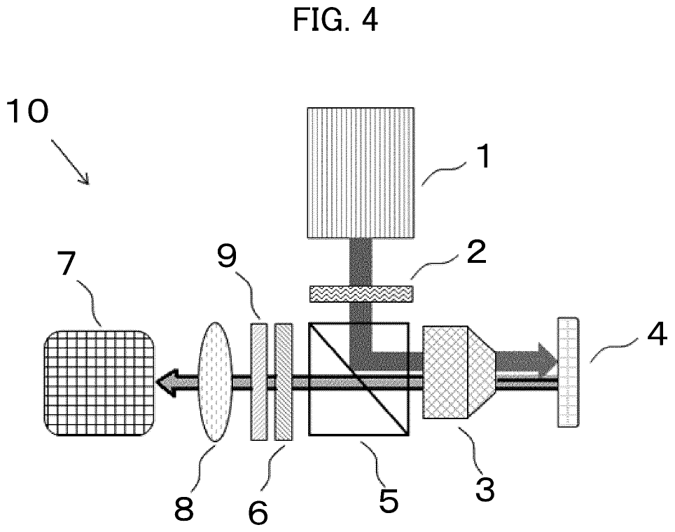

A degree of fluorescence polarization measuring device provided with nine microchannels was used to measure the degree of fluorescence polarization. The configuration of the degree of fluorescence polarization measuring device 10 that was used is illustrated in FIG. 4. The degree of fluorescence polarization measuring device 10 includes a LED light source 1, an excitation filter 2, an objective lens 3, a sample light emitter 4, a dichroic filter 5, a fluorescence filter 6, a digital imaging element (CMOS or CCD) 7, an image forming lens 8, and a liquid crystal element 9. Excitation light having a central wavelength of 565 nm from the LED light source 1 is emitted on the sample in the sample light emitter 4 via the excitation filter 2 and the objective lens 3, the fluorescence emitted by the sample transmits through the dichroic filter 5 and the fluorescence filter 6, and the digital imaging element 7 acquires the transmitted light. The polarization direction of the transmitting fluorescence can be modulated by modulating voltage applied to the liquid crystal element 9 disposed between the fluorescence filter 6 and the image forming lens 8. An image is acquired and calculated by synchronizing this modulation frequency with the capturing frequency of the digital imaging element 7, and the degree of polarization P is extracted as a two-dimensional image.

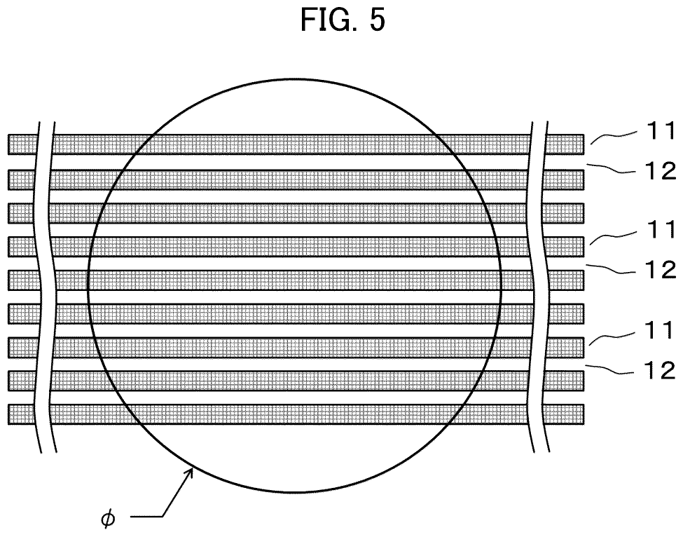

An effective field of view of an optical observation portion of the sample light emitter 4 of the degree of fluorescence polarization measuring device 10 is about 3 mm in diameter. As illustrated in FIG. 5, channels 11 and spaces between channels 12 are provided at equal intervals within the 3 mm diameter of the effective field of view illustrated by the circle. A width of the channels is 200 μm and the space between channels is 100 μm. A depth of the channels is 900 μm. A plurality of samples can be simultaneously measured due to a plurality of microchannels being formed in the sample light emitter 4. An excitation wavelength is 546±11 nm, and a detection wavelength is 590±16.5 nm. Mixed solutions prepared from the nine levels of samples were respectively injected into the nine microchannels, and simultaneously measured.

### Results

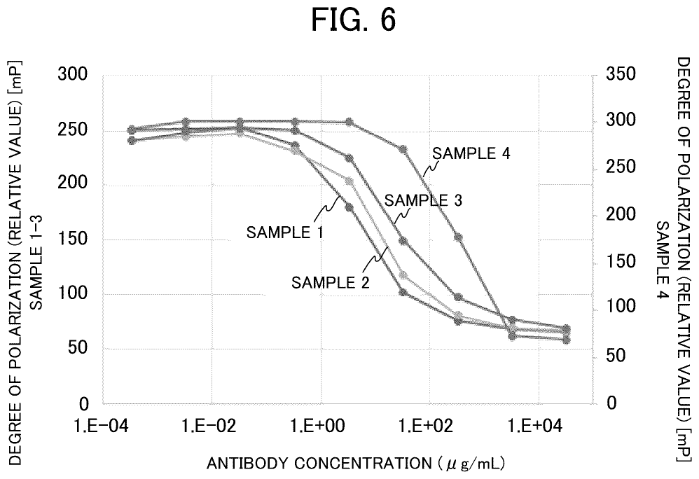

FIG. 6 illustrates the degree of polarization relative to the antigen (histamine) concentration for samples 1 to 4 that have different added amounts of the acylating reagent. The concentration range of the antigen, for which the degree of polarization changes, changed in accordance with the added amount of the acylating reagent. It is demonstrated that the estimable range of the histamine concentration can be expanded by using a plurality of samples having different derivatization rates.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A concentration estimation kit, comprising:
a derivatization reagent that derivatizes at least a portion of an antigen included in a mixed specimen, and that is for obtaining a plurality of samples having different derivatization rates, the derivatization rate being a ratio of a derivatized antigen relative to the antigen included in the specimen;
an antibody that binds to the antigen; and
the antigen modified with a dye.

2. The concentration estimation kit according to claim 1, wherein
the antigen is a hapten,
the antibody is an antibody whose immunogen is a hapten derivative in which an immunogenic substance is bound to the hapten via a linker,
the derivatization reagent imparts, to the hapten in the specimen, a structure identical to at least a portion of the linker, and
the dye binds to the hapten via the structure identical to at least a portion of the linker.

3. The concentration estimation kit according to claim 2, wherein
the hapten is a histamine.

4. The concentration estimation kit according to claim 1, wherein
the derivatization reagent is a plurality of solutions that includes the derivatization reagent at identical concentrations, and that has different volumes.

5. The concentration estimation kit according to claim 1, wherein
the derivatization reagent is a plurality of solutions that includes the derivatization reagent at different concentrations, and that has identical volumes.

6. A concentration estimation method, comprising:
mixing a specimen including an antigen and a derivatization reagent that derivatizes at least a portion of the antigen included in the specimen, and obtaining a plurality of samples having different derivatization rates, the derivatization rate being a ratio of a derivatized antigen relative to the antigen included in the specimen;
mixing each of the samples, an antibody that binds to the antigen, and the antigen modified with a dye to obtain a plurality of solutions to be measured; and
measuring a degree of polarization of each of the solutions to be measured.

7. The concentration estimation method according to claim 6, wherein
the antigen is a hapten,
the antibody is an antibody whose immunogen is a hapten derivative in which an immunogenic substance is bound to the hapten via a linker,
the derivatization reagent imparts, to the hapten in the specimen, a structure identical to at least a portion of the linker, and
the dye binds to the hapten via the structure identical to at least a portion of the linker.

8. The concentration estimation method according to claim 7, wherein
the hapten is a histamine.

9. The concentration estimation method according to claim 6, wherein
the preparing of the samples includes obtaining the plurality of samples by mixing a plurality of solutions that includes the derivatization reagent at identical concentrations, and that has different volumes.

10. The concentration estimation method according to claim 6, wherein
the preparing of the samples includes obtaining the plurality of samples by mixing a plurality of solutions that includes the derivatization reagent at different concentrations, and that has identical volumes.
